(19) United States
(12) Patent Application Publication
(10) Pub. No.: US 2007/0287933 A1
(43) Pub. Date: Dec. 13, 2007

(54) Tissue debulking device and method of using the same

(76) Inventors: Chris Phan, San Leandro, CA (US); Alberto Ruiz Cantu, San Francisco, CA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 11/448,975

(22) Filed: Jun. 8, 2006

(51) Int. Cl. A61B 10/02

(52) U.S. Cl. 600/566; 600/564

(57) ABSTRACT

Apparatuses and methods for debulking a tissue in a patient's body are disclosed herein. In one variation, an apparatus includes a cannula configured to provide percutaneous access to an interior portion of a tissue. The cannula has a distal portion, a proximal portion, and a lumen defined between the distal portion and the proximal portion. A flexible member is coupled to the cannula and configured to steer the distal portion of the cannula within the tissue. An elongate body has a distal portion and is configured to be movably disposed within the lumen of the cannula. The distal portion of the elongate body defines a cutting portion configured to disrupt at least a portion of the tissue when the cutting portion is moved, for example, rotated and/or shuttled. In one variation, the disrupted portion of tissue includes a portion of a tumor.

### BACKGROUND

The invention relates generally to medical devices and procedures, and more particularly to a medical device for accessing percutaneously a tissue and debulking a portion of the tissue.

Known medical devices are configured to access percutaneously a tissue, such as a vertebra or other area of a spine, to perform a variety of different medical procedures. Some known medical devices are configured to remove tissue from within the interior of a vertebra or intervertebral disc. Other known medical devices are configured to provide some type of cutting means to tear or loosen tissue within a vertebra or intervertebral disc.

There are also a variety of medical devices configured to remove a portion of a tumor from within a soft tissue or hard tissue (e.g., bone structure) in the spine or other areas within a patient's body. Some known tumor debulking devices include the use of RF ablation, microwave power, cryotherapy, or laser technology. Some tumors, however, have structural compositions that require a device with sufficient force or power to debulk or cut the tumor so that it can be removed. Many known devices do not provide this required force.

Thus, a need exists for an apparatus and method for debulking a portion of tissue, for example, a tumor, within a hard tissue area or soft tissue area of a patient, having sufficient force to debulk the tissue with minimal damage to the surrounding tissue.

### SUMMARY OF THE INVENTION

Apparatuses and methods for debulking a tissue in a patient's body are disclosed herein. In one variation, an apparatus includes a cannula configured to provide percutaneous access to an interior portion of a tissue. The cannula has a distal portion, a proximal portion, and a lumen defined between the distal portion and the proximal portion. A flexible member is coupled to the cannula and configured to steer the distal portion of the cannula within the tissue. An elongate body has a distal portion and is configured to be movably disposed within the lumen of the cannula. The distal portion of the elongate body defines a cutting portion configured to disrupt at least a portion of the tissue when the cutting portion is moved, for example, rotated and/or shuttled and/or moved in a back-and-forth motion, etc. In one variation, the disrupted portion of tissue includes at least a portion of a tumor.

### BRIEF DESCRIPTION OF THE DRAWINGS

The present invention is described with reference to the accompanying drawings.

FIG. 1 is a schematic illustration of a medical device according to an embodiment of the invention.

FIG. 2 is a side view of a medical device with a partial cut-away portion according to an embodiment of the invention.

FIG. 3 is a cross-sectional view taken along line 3-3 in FIG. 2.

FIG. 4 is a side view of a medical device according to an embodiment of the invention shown inserted into a vertebra.

FIG. 5 is a partial cross-sectional side view of a portion of a medical device according to an embodiment of the invention.

FIG. 6 is an end view taken along line 6-6 in FIG. 5.

FIG. 7 is a side view of a portion of a medical device according to an embodiment of the invention.

FIG. 8 is an end view taken along line 8-8 in FIG. 7.

FIG. 9 is a side view of a portion of a medical device according to an embodiment of the invention.

FIG. 10 is an end perspective view of a portion of a medical device according to an embodiment of the invention.

FIG. 11 is a side view of a portion of a medical device according to an embodiment of the invention.

FIG. 12 is a side view of a portion of a medical device according to another embodiment of the invention.

FIG. 13 is a flowchart illustrating a method according to an embodiment of the invention.

### DETAILED DESCRIPTION

The medical devices described herein are configured for percutaneous deployment within an interior area of a patient's body, such as within a hard tissue area (e.g., bone structure) or soft tissue area of a patient, to debulk, disrupt, sever, and/or cut a portion of a tissue within the tissue area. For example, a medical device according to an embodiment of the invention includes a cutting portion that can debulk, disrupt, sever and/or cut a tissue, or a portion of a tissue, such as tumor, within a tissue area of the patient. In some embodiments, the medical device includes an apparatus having an outer body and an inner body movably disposed within the outer body. The inner body and/or the outer body can be flexible. The apparatus can also include a flexible guide member used to maneuver the apparatus within the tissue area of the patient.

It is also noted that, as used in this specification and the appended claims, the singular forms “a,” “an” and “the” include plural referents unless the context clearly dictates otherwise. Thus, for example, the term “a lumen” is intended to mean a single lumen or a combination of lumens. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body. Thus, for example, the catheter end inserted inside a patient's body would be the distal end of the catheter, while the catheter end outside a patient's body would be the proximal end of the catheter.

In one use of a medical device according to an embodiment of the invention, a portion of a tumor within an interior portion of a tissue, such as a vertebral body, can be debulked, reducing the size of the tumor and weakening its structure so that the debulked portions of the tumor can be removed from the tissue. For some procedures, once the tumor is removed, another procedure can be performed, such as a vertabroplasty or Kyphoplasty procedure on a vertebral body.

In one embodiment, an apparatus includes a cannula configured to provide percutaneous access to an interior portion of a tissue. The cannula has a distal portion, a proximal portion, and a lumen defined between the distal portion and the proximal portion. A flexible member is coupled to the cannula and configured to steer the distal portion of the cannula within the tissue. An elongate body has a distal portion and is configured to be movably disposed within the lumen of the cannula. The distal portion of the elongate body defines a cutting portion. The cutting portion is configured to disrupt at least a portion of the tissue when the cutting portion is moved, for example rotated and/or shuttled in a reciprocating manner.

In another embodiment, an apparatus includes a cannula configured to provide percutaneous access to an interior portion of a tissue. The cannula defines a first lumen configured to be coupled to a suction source, and also defines a second lumen. A flexible member is disposed within the second lumen of the cannula and is configured to maneuver the cannula within the tissue to a location adjacent a tumor. An elongate body has a distal portion and a proximal portion and is configured to be movably disposed within the first lumen of the cannula. The distal portion of the elongate body has a cutting portion configured to disrupt at least a portion of the tumor when the cutting portion is moved. The cannula is configured to remove at least a portion of the tumor through the first lumen of the cannula when the suction source is activated.

In another embodiment, a method is provided that includes percutaneously inserting a distal portion of a device into an interior portion of a tissue. The device includes a cannula and a flexible elongate body movably disposed within a lumen of the cannula. A distal portion of the device is maneuvered within the interior portion of the tissue. The maneuvering includes actuating a proximal portion of a flexible member coupled to the cannula. A cutting portion disposed on a distal portion of the flexible elongate body is moved such that at least a portion of the tissue is disrupted by the cutting portion. The disrupted portion of the tissue can be suctioned from the interior portion of the tissue to a location outside the tissue.

The term “cannula” is used here to mean a component of the apparatus having one or more passageways configured to receive a medical device therethrough and provide percutaneous access to an interior portion of a tissue. For example, the cannula can be substantially tubular. The cannula can be a variety of different shapes and size, such as having a round or octagonal outer and/or inner perimeter.

The term “cutting portion” is used here to mean a portion of a component of the apparatus having at least one cutting surface and being configured to debulk a tissue. The cutting portion can be, for example, a portion of an elongate body configured to be movably disposed within a cannula. The cutting portion can also be, for example, a cutting surface disposed on an outer surface of an elongate body. Examples of a cutting portion can include, a drill configuration, a sharpened tip or edge, multiple cutting surfaces configured in a pattern, a serrated edge, a coring member having a serrated edge, etc.

The term “tissue” is used here to mean an aggregation of similarly specialized cells that are united in the performance of a particular function. For example, a tissue can be a soft tissue area (e.g., a muscle), a hard tissue area (e.g., a bone structure), a vertebral body, an intervertebral disc, a tumor, etc.

The term “debulk” is used here to mean to remove a portion of the bulk of, or reduce the size of a portion of tissue, such as, for example, a tumor within a tissue. As used herein, the terms debulk, disrupt, sever, cut, break-down, etc., are used interchangeably.

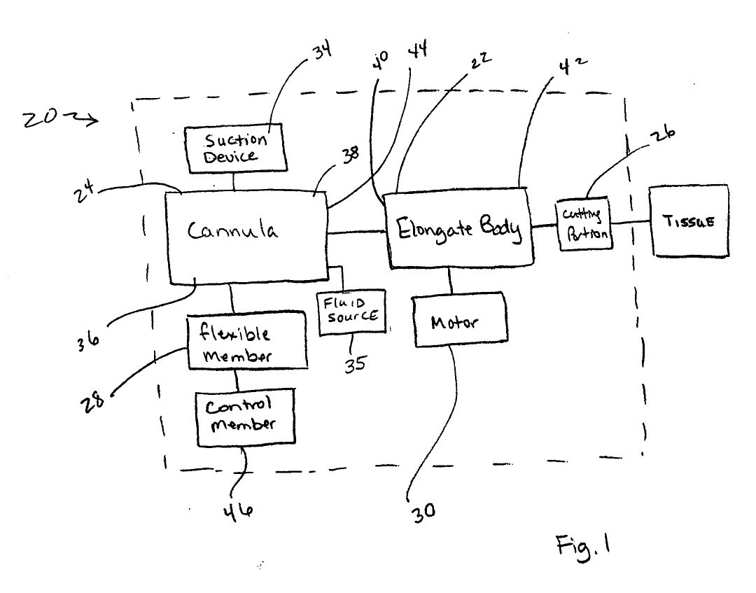

FIG. 1 is a schematic illustration of a medical device according to an embodiment of the invention. A medical device 20 includes a cannula 24 and an elongate body 22 movably disposed within a lumen (not shown in FIG. 1) of the cannula 24. The cannula 24 includes a proximal end portion 36 and a distal end portion 38, and can define one or more lumens (not shown in FIG. 1) between the proximal end portion 36 and the distal end portion 38. The cannula 24 can be flexible such that the cannula 24 is easily maneuverable within a portion of a patient's body.

The elongate body 22 includes a proximal end portion 40, a distal end portion 42, and a cutting portion 26 disposed on or monolithically formed within the distal end portion 42. The cutting portion 26 can include one or more cutting surfaces (not shown in FIG. 1) and have a variety of different configurations, such as, for example, a drill configuration (not shown in FIG. 1). The elongate body 22 can be solid or define a lumen through some or all of the elongate body 22. As with the cannula 24, the elongate body 22 can be flexible for easy maneuverability. For example, in some embodiments, the elongate body 22 includes the cutting portion 26 with the remainder of the elongate body 22 being in the form of a flexible cable or flexible wire.

The medical device 20 can also include a flexible member 28 coupled to the cannula 24. The flexible member 28 can be used to guide and/or maneuver the cannula 24 within the patient's body. The flexible member 28 can be coupled to an exterior portion of the cannula 24 or to an interior portion of the cannula 24, such as within a lumen of the cannula 24. The flexible member 28 can be coupled to the cannula 24 using known attachment methods. In alternative embodiments, the flexible member 28 can be coupled to the elongate body 22 to guide and/or maneuver the elongate body 22 within a tissue.

The cannula 24 can be coupled to a suction device 34, such as a venturi device that is coupled to a suction source (not shown in FIG. 1) via a pressure line (not shown in FIG. 1). The cannula 24 can also be coupled to a source of fluid 35, such that a fluid, such as a saline solution, can flow through the lumen of the cannula 24 to the distal end of the medical device 20 to cool the cutting portion 26 during operation of the medical device 20. A temperature sensor (not shown in FIG. 1) can be optionally coupled to the cannula 24 and/or elongate body 22. The temperature sensor can, for example, provide an indication of the temperature at the tissue site being treated to determine if fluid can be provided to cool the distal end of the medical device 20. A pressure sensor (not shown in FIG. 1) can also be optionally coupled to the cannula and/or elongate body 22. The pressure sensor can, for example, provide an indication of the density of the tissue site to be treated based on the pressure indicated when the pressure sensor contacts the tissue.

The elongate body 22 can be coupled to a motor 30 that is configured to rotate the elongate body 22. The motor 30 and/or the suction device 34 can each be embodied within a hand-held handle assembly (not shown in FIG. 1) or be embodied as a separate component coupled to either the elongate body 22 and/or cannula 24. A control member 46 can be configured to guide the flexible member 28 and can also be coupled to the handle assemble or be embodied in a separate component of the medical device.

In use, the medical device 20 can be percutaneously inserted within a soft or hard tissue area of a patient's body, with the elongate body 22 disposed within a lumen of the cannula 24. For example, for use within a bone structure, the distal end 38 of the cannula 44 can include a trocar tip that can penetrate the bone structure. In some embodiments, the cutting portion 26 of the elongate body 22 can be used to penetrate the tissue. In such an embodiment, the elongate body 22 can extend outside of the cannula 24 while being introduced into the tissue. The flexible member 28 can be used to guide the cannula 24 by maneuvering the control member 46 such that a distal end 44 of the cannula 24 is positioned adjacent to, inserted within, or contacting a selected area within the tissue, such as, for example, a tumor within the tissue area. For example, the control member 46 for the flexible member 28 can cause the flexible member 28 to be pulled in a desired direction, and thus pull the cannula 24 in that direction.

The distal end portion 42 of the elongate body 22 can then be extended outside of the cannula 24 and into the tissue. The motor 30 can actuate the elongate body 22 and cause the cutting portion 26 to rotate and/or shuttle in a back and forth or reciprocating manner, or move side-to-side to cut into and disrupt, debulk or sever at least a portion of the tissue. The suction device 34 can be actuated, either after, or simultaneous with, the movement of the elongate body 22 to suction the disrupted portion or portions of the tissue from within the tissue to a location outside of the patient's body. For example, the portion or portions of the tissue can be suctioned to a collection container (not shown in FIG. 1) for later disposal or analysis. A fluid, such as a saline solution, can also be introduced through the lumen of the cannula to help cool the cutting portion during the debulking, and the excess fluid can be suctioned back through the lumen with the portions of the tissue and into the collection container.

In some embodiments, another medical device, such as a stylet, trocar or bone drill, can be used to penetrate a tissue, such as the cortical bone of a vertebral body, prior to inserting the cannula 24 and elongate body 22. In such a situation, the medical device used to penetrate the bone can provide an access path to the interior of the bone through which the cannula 24 and elongate body 22 can be inserted. In some embodiments, after such an access path is formed, only the elongate body 22 is inserted through the access path and maneuvered to a desired location within the bone.

Thus, in any of the embodiments described herein, the flexibility of the elongate body 22 and the cannula 24 allows components of the medical device 20 to be steered within a tissue to a desired treatment site within the tissue. The following examples describe various embodiments of the medical device 20 with reference to use within a vertebra to illustrate various aspects of the invention. However, one of ordinary skill in the art having the benefit of this disclosure would appreciate that the medical devices described herein can be used on other areas within a patient's body as well.

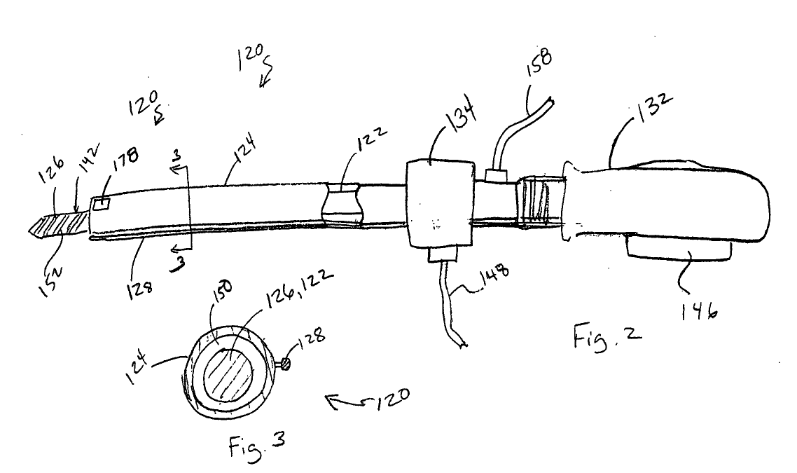

FIGS. 2 and 3 illustrate a medical device according to an embodiment of the invention. A medical device 120 includes a cannula 124 and an elongate body 122 movably disposed within a lumen 150 of the cannula 124. In this embodiment, the cannula 124 has a single lumen 150. A flexible member 128 is coupled to an exterior surface of the cannula 124. The cannula 124 is coupled to a suction device 134 and a fluid source (not shown) through a fluid line 158. As stated previously, the suction device 134 can be coupled to a suction source (not shown) via a pressure line 148. A temperature sensor 178 coupled to the cannula 124 can provide an indication of the temperature at the treatment site. The fluid source can be configured such that when the temperature at the treatment site reaches a threshold temperature, the fluid (e.g., saline solution) can be automatically introduced into the lumen 150 of the cannula 124 and to the treatment site. For example, the fluid source can include a control system that receives temperature data from the temperature sensor and dispenses fluid from the fluid source based on the temperature data.

The elongate body 122 includes a cutting portion 126 disposed on a distal end portion 142. In this embodiment, the cutting portion 126 includes a plurality of cutting surfaces 152 in a drill configuration, and can be used to disrupt, debulk or sever a portion of a tissue, such as a tumor. The elongate body 122 and the cannula 124 are coupled to a handle assembly 132. The handle assembly 132 can include a motor (not shown) to rotate the elongate body 122, and a control member 146 to control the movement of the flexible member 128.

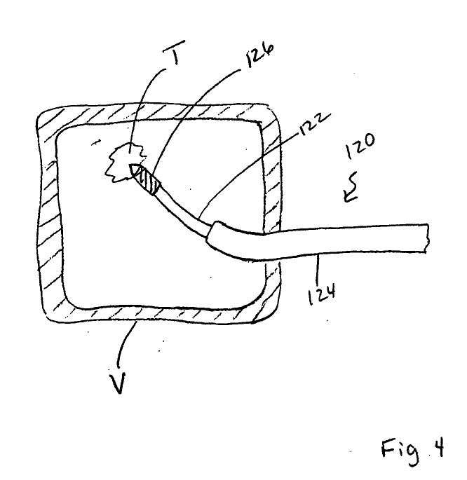

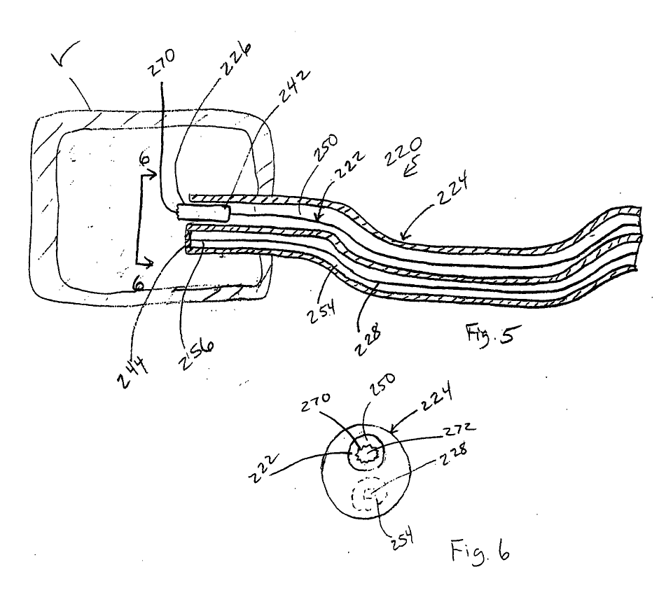

As shown in FIG. 4, the medical device 120 can be percutaneously inserted into a tumor T within a vertebra V. The elongate body 122 can be rotated via the motor, such that the cutting portion 126 disrupts, severs, or debulks at least a portion of the tumor T. The debulked portion(s) of the tumor T can be simultaneously removed from the vertebra via suction through the lumen 150 of the cannula 124 using the suction device 134. Simultaneously, or sequentially with the cutting portion 126 disrupting the tumor T, fluid such as a saline solution can be dispensed from the fluid source, through the fluid line 158 and the lumen 150 of the cannula 124 to cool the cutting portion 126 and/or the tissue treatment site.

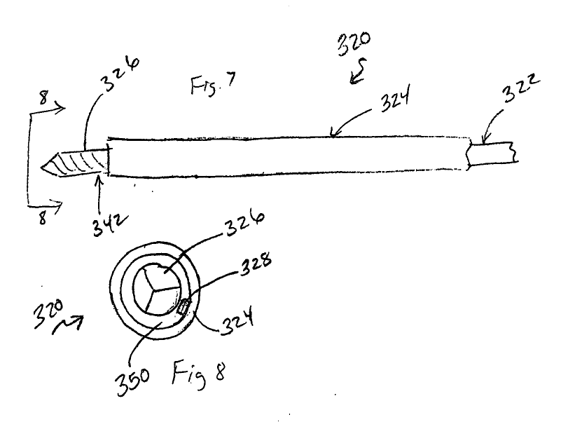

FIG. 5 illustrates a cross-sectional view of a portion of a medical device according to another embodiment of the invention shown inserted within a vertebra V; FIG. 6 is a cross-sectional view of the medical device shown in FIG. 5 taken along line-6-6. The medical device 220 includes a cannula 224 and an elongate body 222 movably disposed within a first lumen 250 of the cannula 224. A cutting portion 226 is disposed at a distal end portion 242 of the elongate body 222. In this embodiment, the cutting portion is in the form of a coring member having a serrated edge 270 defining an opening 272.

In this embodiment, the cannula 224 also includes a second lumen 254, through which a flexible member 228 is disposed. Although a distal end 256 of the flexible member 228 is shown coupled to a distal end 244 of the cannula 224, in other embodiments, the flexible member 228 can be coupled to the cannula 224 at other locations on the cannula 224, such as to a side wall of the cannula 224. As with the previous embodiments, the cannula 224 and the elongate body 222 can be coupled to a handle assembly (not shown) having a motor (not shown) to control the rotational and/or translational movement of the elongate body 222, and a control member (not shown) to control movement of the flexible member 228. A suction source (not shown) can also be coupled to the cannula 224 to provide suction through the first lumen 250, and a fluid source (not shown) can be coupled to the cannula 324 to provide a fluid to cool the cutting member 326.

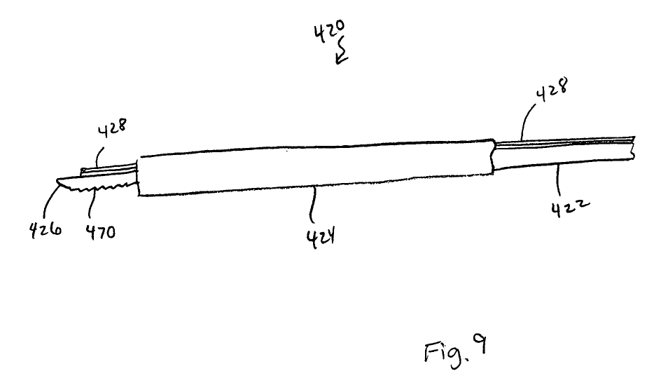

Another medical device according to an embodiment of the invention is illustrated in FIGS. 7 and 8. A medical device 320 includes a cannula 324 defining a lumen 350, and an elongate body 322 movably disposed within the lumen 350. As with the previous embodiments, the elongate body 322 includes a cutting portion 326 disposed at a distal end portion 342 configured to disrupt, debulk, cut, or sever at least a portion of a tissue, such as a tumor portion of the tissue. The medical device 350 is similar to the previous embodiments and performs substantially the same functions as the previous embodiments, except in this embodiment, a flexible member 328 is coupled to an interior sidewall of the cannula 324 within the lumen 350.

As with the previous embodiments, the cannula 324 and the elongate body 322 can be coupled to a handle assembly (not shown) having a motor (not shown) to actuate and control movement of the elongate body 322, and a control member (not shown) to actuate and control movement of the flexible member 328. A suction source (not shown) can also be coupled to the cannula 324 to provide suction through the lumen 350, and a fluid source (not shown) can be coupled to the cannula 324 to provide a fluid to cool the cutting member 326.

FIG. 9 illustrates yet another embodiment of a medical device. In this embodiment, a medical device 420 includes a cannula 424 and an elongate body 422. A cutting portion 426 in the form of a blade with a serrated edge 470 is disposed on a distal end portion of the elongate body 422. The elongate body 422 can be actuated to move, for example, in a reciprocating or back-and-forth manner. A flexible member 428 is coupled to the elongate body 424 to help maneuver the elongate body 422 within a tissue.

As with the previous embodiments, the cannula 424 and the elongate body 422 can be coupled to a handle assembly (not shown) having a motor (not shown) to control the rotational and/or translational movement of the elongate body 422, and a control member (not shown) to actuate movement of the flexible member 428. A suction source (not shown) can also be coupled to the cannula 424 to provide suction through a lumen (not shown) of the cannula 424, as well as a fluid source (not shown).

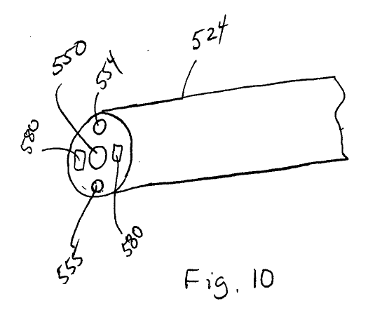

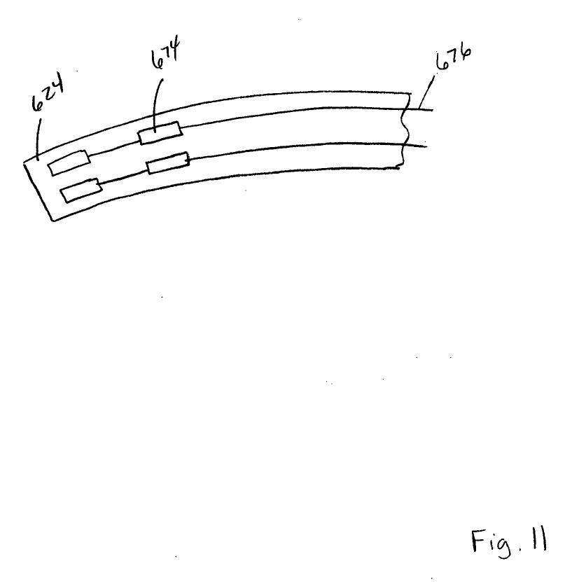

FIG. 10 illustrates an alternative embodiment of a cannula that can be used with a medical device. A cannula 524 defines a first lumen 550, a second lumen 554, and a third lumen 555. An elongate body as described herein, or another medical device can be received within the first lumen 550. A flexible member (not shown) can be coupled to the cannula 524 within each of the second lumen 554 and the third lumen 555. For example, to turn the cannula 524 in a first direction, the user can pull on one of the flexible members, and to turn the cannula 524 in an opposite direction, the user can pull the other of the flexible members. Alternatively, a user can pull one and push the other of the flexible members to turn the cannula in one direction, and vice versa to turn in the opposite direction. A pair of pressure sensors 580 are coupled to a distal end of the cannula 524. As stated previously, the pressure sensors 580 can be used to determine a density or hardness of the tissue to be treated. For example, the cannula 524 can be pushed into contact with the tissue to be treated and pressure reading obtained. Based on the pressure reading, a determination can be made as to what type of elongate body or other medical device should be used to treat the tissue.

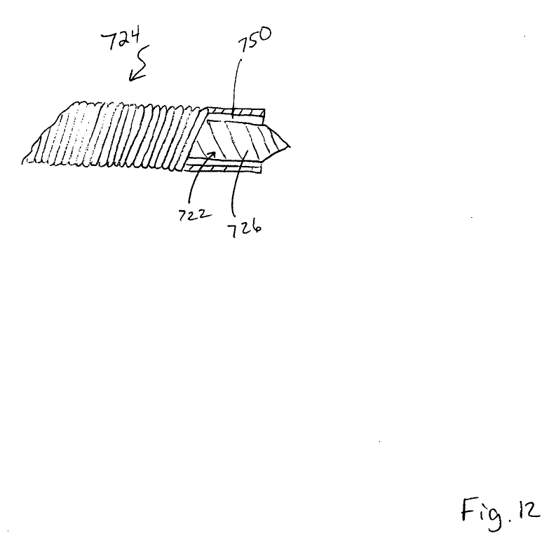

FIG. 11 illustrates an alternative embodiment of a device for maneuvering or steering a cannula and/or elongate body. In this embodiment, multiple micro-actuators 674 are coupled to an outer surface of a cannula 624. Four micro-actuators 674 are illustrated in the side-view of FIG. 11, but any number of micro-actuators 674 can be used and disposed about the outer surface of cannula 624. The micro-actuators 674 can be coupled via a wire 676 to an actuator (not shown) configured to supply an electric charge to selected ones of the micro-actuators 674 to cause the cannula 624 to move in a particular direction. The micro-actuators 674 can be any type of material that expands or contracts when an electrical current is applied or charged.

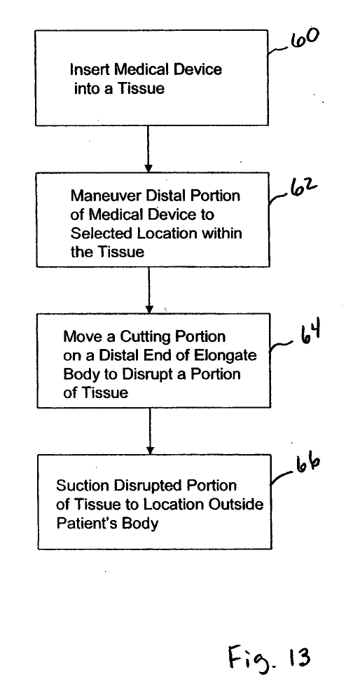

FIG. 12 illustrates a cannula according to another embodiment of the invention. A cannula 724 is formed with a torsion spring that defines a lumen 750. An elongate body 722 having a cutting portion 726, can be received within the lumen 750 of cannula 724. As with the previous embodiments described herein, another elongate body as described herein, or another medical device can alternatively be received within the lumen 750 of the cannula 724. The torsion spring cannula 724 can be moved in a particular direction by applying a torque to the cannula 724 in a desired direction. Thus, the cannula 724 can bend and/or curve to allow it to be maneuvered within a tissue such that the distal end portion of the medical device can be positioned in the tissue as desired. Alternatively, a flexible member as described previously can be coupled to the cannula 724 and used to maneuver the cannula 724.

The various components of a medical device as described herein can be provided in a kit. For example, one or more cannulas, one or more elongate bodies, and/or one or more flexible members can be provided. A user can select the particular cannula, elongate body or flexible member (or other steering device) to use for the particular medical procedure to be performed.

FIG. 13 is a flowchart illustrating a method of using a medical device according to an embodiment of the invention. A method includes at 60, percutaneously inserting a distal portion of a medical device into an interior portion of a tissue within a patient's body. The tissue can be, for example, a vertebral body. The medical device includes a cannula and a flexible elongate body movably disposed within a lumen of the cannula. In some embodiments, the cannula is also flexible. A distal portion of the medical device is maneuvered to a selected treatment location within the interior portion of the tissue at 62. The maneuvering can include actuating a proximal portion of a flexible member coupled to the cannula. For example, the flexible member can be tensioned and/or relaxed to direct the cannula to a desired location within the tissue. In some embodiments, the flexible member is elongate, and is coupled to the cannula such that the cannula and the flexible member are in a side-by-side relationship.

At 64, a cutting portion disposed on a distal portion of the flexible elongate body is moved such that at least a portion of the tissue is disrupted by the cutting portion. For example, the cutting portion can be disposed outside of the cannula and within the interior of the tissue and rotated. In some embodiments, a tumor within the tissue is disrupted. In some embodiments, the disrupted portion of the tissue is suctioned to a location outside of the patient's body. The suctioning can, in some cases, be performed simultaneously with the movement of the cutting portion.

The medical device for any of the embodiments may be constructed with any suitable material used for such a medical device. For example, the cannula, the elongate body and the flexible member can be constructed with a suitable biocompatible material, such as various biocompatible metal or plastic materials (e.g., various polymers) that are structured so as to provide flexible characteristics. The cutting member can likewise be constructed with suitable biocompatible metals or plastics.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, the medical device can include various combinations of the components described in the various embodiments. In some embodiments, the medical device may not include a source of suction power or source of fluid. In some embodiments, the cannula can have more than one, two or three lumens as illustrated herein. In addition, as stated previously, the medical device is not limited to use within a vertebra and can be used to disrupt, sever, cut, or debulk a portion of a tissue within another tissue area within a patient's body. In addition, other medical devices can be used in conjunction with one or more of the components described herein. For example, an elongate body can be used with other types of cannulas. A steerable cannula as described herein can be used to provide access to a tissue for use with other medical devices, such as a fiber optic scope, an ultrasound device, or an RF ablation device. The flexible member and other steering devices described herein can also be used with other cannulas and medical devices, not specifically described. Other types of cutting methods can also be used with the medical devices and methods described herein. For example, an RF electrode or ultrasonic device can be used to cut or debulk instead of the specific cutting portions described herein.

What is claimed is:

1. An apparatus, comprising:
a cannula configured to provide percutaneous access to an interior portion of a tissue, the cannula having a distal portion, a proximal portion, and a lumen defined between the distal portion and the proximal portion;
a flexible member coupled to the cannula and configured to steer the distal portion of the cannula within the tissue; and
an elongate body having a distal portion and configured to be movably disposed within the lumen of the cannula,
the elongate body having a cutting portion disposed at the distal portion of the elongate body, the cutting portion configured to disrupt at least a portion of the tissue when the elongate body is moved relative to the cannula.

2. The apparatus of claim 1, wherein the lumen of the cannula is a first lumen, the cannula further defining a second lumen, the apparatus further comprising:
a suction source coupled to the second lumen of the cannula, the cannula configured to remove the disrupted portion of the tumor through the second lumen of the cannula when the suction source is activated,

3. The apparatus of claim 1, wherein the elongate body is flexible.

4. The apparatus of claim 1, wherein the lumen of the cannula is a first lumen, the cannula further defining a second lumen, the flexible member disposed within the second lumen of the cannula.

5. The apparatus of claim 1, wherein the cutting portion includes a plurality of cutting surfaces.

6. The apparatus of claim 1, wherein the cutting portion is configured to be disposed outside the cannula and within an interior portion of the tissue.

7. The apparatus of claim 1, wherein the cannula is configured to be coupled to a suction source, the cannula configured to remove the disrupted portion of tissue through the lumen of the cannula when the suction source is activated.

8. The apparatus of claim 1, wherein the cutting portion is configured to cut cancellous bone within a vertebral body.

9. The apparatus of claim 1, wherein the tissue is a vertebral body.

10. The apparatus of claim 1, wherein the cannula is flexible.

11. The apparatus of claim 1, wherein the tissue is a bone structure.

12. The apparatus of claim 1, wherein the disrupted tissue includes at least a portion of a tumor.

13. The apparatus of claim 1, wherein the lumen is a first lumen, the flexible member is a first flexible member, the cannula further defines a second lumen and a third lumen, the apparatus further comprising:
a second flexible member, the first flexible member disposed within the second lumen, the second flexible member disposed within the third lumen, the first flexible member and the second flexible member configured to steer the distal portion of the cannula within the tissue.

14. An apparatus, comprising:
a cannula configured to provide percutaneous access to an interior portion of a tissue, the cannula defining a first lumen and a second lumen, the first lumen configured to be coupled to a suction source;
a flexible member disposed within the second lumen of the cannula, the flexible member configured to maneuver the cannula within the vertebral body to a location adjacent a tumor; and
an elongate body having a distal portion and a proximal portion and configured to be movably disposed within the first lumen of the cannula,
the distal portion of the elongate body having a cutting portion, the cutting portion configured to disrupt at least a portion of the tumor when the distal portion of the elongate body is moved relative to the cannula, the cannula configured to remove the at least a portion of the tumor that is disrupted through the first lumen of the cannula when the suction source is activated.

15. The apparatus of claim 14, wherein the elongate body is flexible and configured to be maneuvered through the lumen of the cannula.

16. The apparatus of claim 14, wherein the cutting portion of the elongate body includes a plurality of cutting surfaces.

17. The apparatus of claim 14, wherein the flexible member is remotely actuatable.

18. The apparatus of claim 14, wherein the cutting portion of the elongate body is configured to be disposed outside the cannula and within the interior portion of the tissue.

19. The apparatus of claim 14, wherein the cutting portion of the elongate body is configured to cut cancellous bone within a vertebral body.

20. The apparatus of claim 14, wherein the tissue is a vertebral body.

21. The apparatus of claim 14, wherein the cannula is flexible.

22. A method, comprising:
percutaneously inserting a distal portion of a device into an interior portion of a tissue within a patient's body, the device including a cannula and a flexible elongate body movably disposed within a lumen of the cannula;
maneuvering a distal portion of the device to a selected location within the interior portion of the tissue, the maneuvering including actuating a flexible member coupled to the cannula; and
moving a cutting portion disposed on a distal portion of the flexible elongate body such that at least a portion of the tissue is disrupted by the cutting portion.

23. The method of claim 22, further comprising:
suctioning the disrupted portion of tissue to a location outside of the patient's body.

24. The method of claim 22, wherein the moving is performed simultaneously with the suctioning the disrupted portion of tissue to a location outside of the patient's body.

25. The method of claim 22, wherein the maneuvering includes at least one of tensioning or relaxing the flexible member.

26. The method of claim 22, wherein the flexible member is elongate, the flexible member is coupled to the cannula such that the cannula and the flexible member are in a side-by-side relationship.

27. The method of claim 22, wherein the cannula is flexible.

28. The method of claim 22, further comprising:
moving the flexible elongate body such that the cutting portion is disposed outside the cannula and within the interior portion of the tissue.

29. The method of claim 22, wherein the moving the cutting portion includes rotating the cutting portion.

30. An apparatus, comprising:
an elongate body having a cutting portion disposed at a distal end portion; and
a flexible guide member coupled to the elongate body, the flexible guide member configured to maneuver the elongate body within a tissue to position the cutting portion at a selected treatment site within the tissue, the cutting portion configured to disrupt at least a portion of the tissue at the treatment site.

31. The apparatus of claim 30, further comprising:
a cannula defining a lumen, the elongate body configured to be movably disposed within the lumen of the cannula.

32. The apparatus of claim 30, wherein the cutting portion has a drill configuration.

33. The apparatus of claim 30, wherein the cutting portion has a coring configuration having a serrated edge.

34. The apparatus of claim 30, wherein the cutting portion includes a blade having a serrated edge.

35. The apparatus of claim 30, further comprising:
a cannula having a torsion spring configuration and defining a lumen, the elongate body movably disposable within the lumen of the cannula.

36. The apparatus of claim 30, further comprising:
a cannula defining a lumen, the elongate body movably disposable within the lumen of the cannula; and
a pressure sensor coupled to the cannula, the pressure sensor configured to indicate a pressure exerted on the cannula by the tissue.

37. The apparatus of claim 30, further comprising:
a cannula defining a lumen, the elongate body movably disposable within the lumen of the cannula; and
a plurality of micro-actuators coupled to the cannula configured to move the cannula when a selected micro-actuator from the plurality of micro-actuators is charged with an electric current.

38. The apparatus of claim 30, wherein the elongate body is flexible.
